(19) United States
(12) Patent Application Publication
(10) Pub. No.: US 2014/0235959 A1
(43) Pub. Date: Aug. 21, 2014

(54) METHODS AND ALGORITHMS FOR SUPERVISORY CLOSED-LOOP DETERMINATION OF OPTIMIZED SCHEDULING OF VENTILATOR WEANING TRIALS

(71) Applicant: Covidien LP, Mansfield, MA (US)

(72) Inventors: Mehdi Jafari, Laguna Hills, CA (US); Peter Doyle, Vista, CA (US)

(73) Assignee: Covidien LP, Mansfield, MA (US)

(21) Appl. No.: 13/768,318

(22) Filed: Feb. 15, 2013

(51) Int. Cl. A61B 5/00; A61B 5/1455; A61B 5/0205; A61M 16/08; A61B 5/22; A61M 16/04; A61M 16/06; A61M 16/00; A61B 5/0476

(52) U.S. Cl. CPC A61B 5/4836; A61M 16/0057; A61B 5/14551; A61B 5/0205; A61B 5/0476; A61B 5/224; A61B 5/4848; A61M 16/04; A61M 16/06; A61M 16/0875; A61M 16/0816
USPC 600/301; 600/300; 128/204.23; 600/532; 600/324; 600/544; 600/481; 600/533; 600/587

(57) ABSTRACT

Ventilatory systems and methods are disclosed for detecting weaning readiness of a patient. In one aspect the method includes the steps of collecting patient data; analyzing the collected patient data to generate at least one patient parameter; comparing the at least one patient parameter to predetermined weaning readiness threshold criteria; and providing an advisory proposition to a clinician indicating a readiness of the patient to start a weaning trial if the at least one patient parameter meets the weaning readiness threshold criteria. The weaning trial may additionally or alternatively be automatically executed if the at least one patient parameter meets the weaning readiness threshold criteria.

### TECHNICAL FIELD

The present disclosure relates generally to medical devices and to methods and algorithms for ventilator weaning.

### BACKGROUND

A ventilator is a device that mechanically helps patients breathe by replacing some or all of the muscular effort required to inflate and deflate the lungs. During prolonged mechanical ventilation (PMV) a patient often becomes partially or totally reliant on a ventilator to continue breathing and becomes unable to breathe alone. Current techniques for determining fitness for weaning the patient require the clinician to be alert to improvements in the patient that indicate a readiness to be weaned off of the ventilator. If a clinician is inattentive, the duration of the PMV is unnecessarily increased, and if over-aggressive the patient can suffer a setback in their recovery.

### SUMMARY

In accordance with an aspect of the present disclosure, a method of detecting weaning readiness of a patient is disclosed including the steps of collecting patient data; analyzing the collected patient data to generate at least one patient parameter; comparing the at least one patient parameter to predetermined weaning readiness threshold criteria; and providing an advisory proposition to a clinician indicating a readiness of the patient to start a weaning trial if the at least one patient parameter meets the weaning readiness threshold criteria. The weaning trial may additionally or alternatively be automatically executed if the at least one patient parameter meets the weaning readiness threshold criteria.

The present disclosure provides new and unique advantages to ventilator treatment over prior ventilator systems. By automatically analyzing collected patient data from both internal and external sources and generating patient parameters related to the weaning readiness of a patient, the ventilatory system is able to provide a clinician with increased information and decision making capabilities while reducing the amount of time the clinician must spend by the patient's bedside performing assessments and analysis to determine whether a patient is ready to undergo weaning trials. By providing the clinician with advisory propositions in addition to the collected patient data, the ventilatory system is able to further assist the clinician and increase the clinician's efficiency by removing a majority of the guesswork and analysis involved in making determinations of patient weaning readiness. This provides an added benefit in short staffing situations by reducing the amount of time a clinician must stay by the patient's bedside to assess the patient and provides an increased confidence to the clinician of when weaning trials are appropriate. Finally, providing both automated weaning readiness determination and automated weaning trial execution provides a hospital and/or clinician with the added benefit of having patients weaned off of ventilator treatment without any direct intervention.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

### BRIEF DESCRIPTION OF THE DRAWINGS

The present disclosure and its various aspects and features are described hereinbelow with reference to the accompanying drawings, wherein:

FIG. 1 is a diagram illustrating an embodiment of a ventilator connected to a patient;

FIG. 2 is a block diagram illustrating an embodiment of a ventilatory system, in accordance with an aspect of the present disclosure;

FIG. 3 is a flowchart illustrating a method of determining weaning readiness, in accordance with an aspect of the present disclosure.

The figures depict preferred embodiments of the present disclosure for purposes of illustration only. One skilled in the art will readily recognize from the following discussion that alternative embodiments of the structures and methods illustrated herein may be employed without departing from the principles of the present disclosure described herein.

### DETAILED DESCRIPTION

Although the present disclosure will be described in terms of a specific embodiment, it will be readily apparent to those skilled in this art that various modifications, rearrangements and substitutions may be made without departing from the spirit of the present disclosure. The scope of the present disclosure is defined by the claims appended hereto.

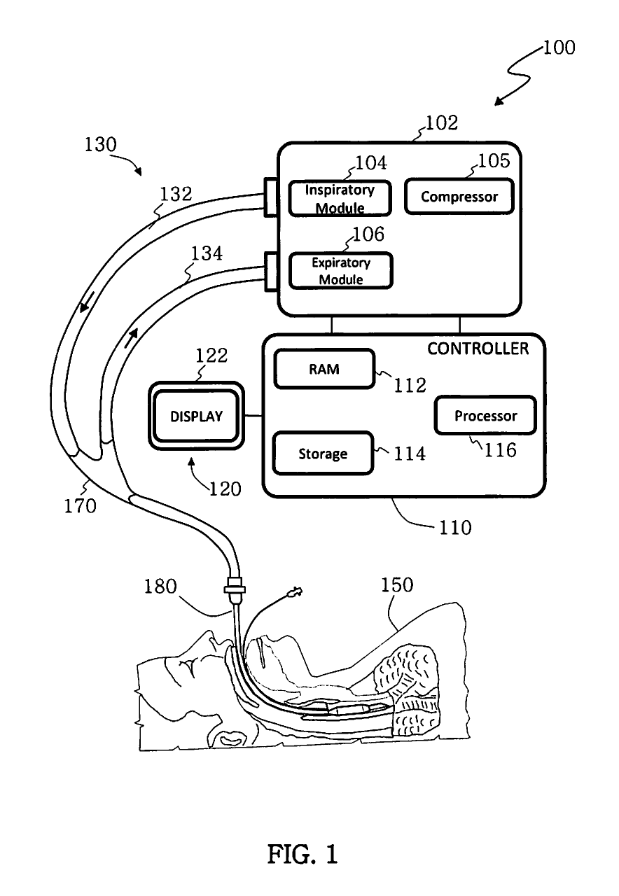

FIG. 1 is a diagram illustrating a ventilator 100 connected to a patient 150. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface 180.

Ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, breath types, view monitored parameters, etc.). Controller 110 may include memory 112, one or more processors 116, memory 114, and/or other components of the type commonly found in command and control computing devices. In the depicted example, operator interface 120 includes a display 122 that may be touch-sensitive and/or voice-activated, enabling the display 122 to serve both as an input and output device. Alternatively, a keyboard (not shown) or other data input device may be employed.

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network via wired or wireless means.

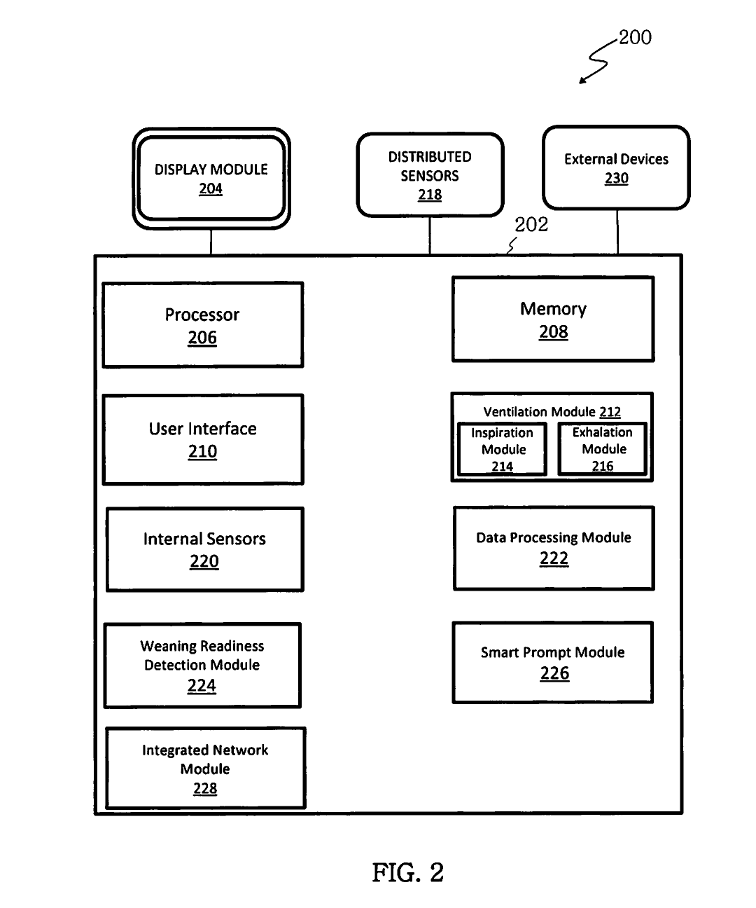

FIG. 2 is a block-diagram illustrating an embodiment of a ventilatory system 200 for monitoring and evaluating ventilatory parameters associated with a ventilator weaning trials.

Ventilatory system 200 includes ventilator 202 with its various modules and components. That is, ventilator 202 may further include, for example, memory 208, one or more processors 206, user interface 210, and ventilation module 212 (which may further include an inspiration module 214 and an exhalation module 216). Memory 208 is defined as described above for memory 112. Similarly, the one or more processors 206 are defined as described above for one or more processors 116. Processors 206 may further be configured with a clock whereby elapsed time may be monitored by the ventilatory system 200. The ventilatory system 200 may also include a display module 204 communicatively coupled to ventilator 202. Display module 204 provides various input screens, for receiving clinician input, and various display screens, for presenting useful information to the clinician. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., including ventilatory data, alerts, patient information, parameter settings, etc.). The elements may include controls, graphics, charts, tool bars, input fields, smart prompts, etc. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed to the clinician in the form of graphs, wave representations, pie graphs, or other suitable forms of graphic display. For example, one or more smart prompts may be displayed on the GUI and/or display module 204 upon determination that a patient is ready to undergo weaning trials. Additionally or alternatively, one or more smart prompts may be communicated to a remote monitoring system coupled via any suitable means to the ventilatory system 200.

As used herein the term “clinician” refers to any medical professional (i.e., doctor, surgeon, nurse, or the like) performing and/or monitoring and/or supervising a medical procedure involving the use of the embodiments described herein.

Ventilation module 212 may oversee ventilation of a patient according to prescribed ventilatory settings. For example, ventilation of a patient may be performed according to a series of ventilation parameters and/or ventilatory data based on a series of controlling equations. By way of general overview, the basic elements impacting ventilation may be described by the Equation of Motion as described in co-pending application Ser. No. 13/035,974, the entirety of which is incorporated herein by reference. Likewise the measurement and calculation of various parameters including, for example, pressure, flow and volume, respiratory compliance, respiratory resistance, pulmonary time constant, and normal resistance and compliance, are also described in co-pending application Ser. No. 13/035,974.

The ventilatory system 200 may also include one or more distributed sensors 218 communicatively coupled to ventilator 202. Distributed sensors 218 may communicate with various components of ventilator 202, e.g., processor 206, ventilation module 212, internal sensors 220, data processing module 222, a weaning readiness detection module 224, and any other suitable components and/or modules. Distributed sensors 218 may detect changes in ventilatory parameters indicative of a patient's potential readiness to undergo weaning trials. Distributed sensors 218 may be placed in any suitable location, e.g., within the ventilatory circuitry or other devices communicatively coupled to the ventilator. For example, sensors may be affixed to the ventilatory tubing or may be imbedded in the tubing itself. According to some embodiments, sensors may be provided at or near the lungs (or diaphragm) for detecting a pressure in the lungs. Additionally or alternatively, sensors may be affixed or imbedded in or near wye-fitting 170 and/or patient interface 180, as described above.

Distributed sensors 218 may further include pressure transducers that may detect changes in circuit pressure (e.g., electromechanical transducers including piezoelectric, variable capacitance, or strain gauge). Alternatively or additionally, sensors may utilize optical or ultrasound techniques for measuring changes in ventilatory parameters. A patient's blood parameters or concentrations of expired gases may also be monitored by sensors to detect physiological changes that may be used as indicators to study physiological effects of ventilation. The results of such studies may be used for diagnostic or therapeutic purposes. Any distributed sensory device useful for monitoring changes in measurable parameters during ventilatory treatment may be employed in accordance with embodiments described herein.

Ventilator 202 may further include one or more internal sensors 220. Similar to distributed sensors 218. Internal sensors 220 may communicate with various components of ventilator 202, e.g., processor 206, ventilation module 212, internal sensors 220, data processing module 222, a weaning readiness detection module 224, and any other suitable components and/or modules. Internal sensors 220 may employ any suitable sensory or derivative technique for monitoring one or more parameters associated with the ventilation of a patient. The one or more internal sensors 220 may be placed in any suitable internal location, such as, for example, within the ventilatory circuitry or within components or modules of ventilator 202. For example, sensors may be coupled to the inspiratory and/or expiratory modules for detecting changes in, for example, circuit pressure and/or flow. Additionally or alternatively, internal sensors 220 may utilize optical or ultrasound techniques for measuring changes in ventilatory parameters. For example, a patient's expired gases may be monitored by internal sensors 220 to detect physiological changes indicative of the patient's condition and/or readiness for weaning. Internal sensors 220 may employ any suitable mechanism for monitoring parameters of interest in accordance with embodiments described herein.

Ventilator 202 may further include an integrated network module 228 for communicating with external devices 230 that are integrated or networked with ventilator 202. For example, ventilator 202 may operate as an external processing platform for data received from external devices 230. External devices 230 may provide data to ventilator 202 including, for example, end-tidal CO₂, SpO₂, levels of sedation, brainwave activity, body weight, blood pressure, blood oxygen level, etc. Some examples of external devices include capnometers, pulse oximeters, brain function monitors and hemodynamic monitors. Alternatively, an external device 230 such as a server or central processing device (not shown) may receive ventilatory data or parameters from ventilator 202, distributed sensors 218, internal sensors 220, and/or other external devices 230, perform necessary processing on the received data, and transmit the results of the processing to ventilator 202 for use by ventilator 202. For example, processor 206 may be located remotely from ventilator 202 and may be disposed on an external device 230 such as a server or central processing device (not shown). In addition, ventilation module 212, data processing module 222, weaning readiness detection module 224, and/or any other suitable components and/or modules may also be located remotely from ventilator 202 and may, for example, be disposed on an external device 230 such as a server or other central processing device. The server or central processing device, for example, may determine when a patient is ready to undergo a weaning trial based on the received patient data and parameters, and instruct the ventilator 202 to perform the weaning trial. The server or central processing device may also or alternatively instruct ventilator 202 to perform one or more maneuvers as described in more detail below.

Weaning readiness detection module 224 is configured to perform a supervisory closed-loop determination of a patient's readiness to undergo ventilator weaning trials, also known as spontaneous breathing trials. Weaning readiness detection module 224 may run on ventilator 202, for example, by executing a software routine on processor 206. Weaning readiness detection module 224 may, for example, execute the software routine in the background as part of the ventilator software operating in real time or quasi-real time on ventilator 202 during ventilation. Alternatively, weaning readiness detection module 224 may instead be run as an application on a processor of an external device 230, e.g., a central server or a multiple medical device management system, and may communicate and control ventilator 202 via, for example, a wired, wireless or other similar connection.

When determining whether a patient is ready for a weaning trial, a number of factors are taken into account. Weaning readiness detection module 224 collects available data from one or more sources, e.g., ventilator 202 and/or external devices 230 and causes processor 206 to analyze and/or trend the collected data. Weaning readiness detection module 224 also may provide an advisory proposition to the clinician of a readiness for weaning trials via an output device, for example, display module 204, user interface 210, smart prompt module 226, a hand held device, a nursing station, a hospital server, a central monitoring station and/or another location set by a clinician. For example, the advisory proposition may be output by the output device in the form of a visual, audio, or other similar type of output capable of conveying to the clinician the patient's readiness for weaning trials. Weaning readiness detection module 224 may also or alternatively execute a weaning trial automatically and may provide the clinician with an advisory proposition upon successful completion of the weaning trial, e.g., spontaneous breathing for more than a predetermined amount of time such as, for example, half an hour, an hour, etc., indicating that removal of ventilation tubing system 130 and termination of ventilation is appropriate. Alternatively, if a weaning trial fails, weaning readiness detection module 224 or ventilator 202 may automatically implement standard ventilation and notify the clinician of the failure.

Weaning readiness detection module 224 collects, analyzes and trends internal patient data from distributed sensors 218, internal sensors 220, memory 208, or other data generated by ventilatory system 200 that relates to the operation of ventilator 202 or to the status of a patient during ventilation, and determines whether a patient is ready for a weaning attempt. For example, weaning readiness detection module 224 may base a determination of weaning readiness on internal patient data such as measured or calculated respiratory mechanics and/or measured or calculated indices of muscle strength. Examples of measured or calculated respiratory mechanics include airway pressure, flow, volume, compliance (C), resistance (R), respiratory frequency, tidal volume, P_{0.1} (pressure in the first 100 ms of inspiration as an indicator of respiratory drive), work of breathing, rapid shallow breathing index (RSBI), etc. Examples of measured or calculated indices of muscle strength include Vital Capacity (VC), maximum inspiratory or expiratory pressures (MIP, MEP), trigger pressure, Maximum minute volume (MMV), etc.

Weaning readiness detection module 224 may also initiate various maneuvers to assist in the determination of respiratory mechanics or indices of muscle strength or respiratory drive such as, for example, a P_{0.1} maneuver (Occlusion Pressure), a Vital Capacity (VC) maneuver, a Negative Inspiratory Force (NIF) maneuver, a Maximum minute volume (MMV) maneuver or other maneuvers that may provide valuable indices for the evaluation of muscle strength or respiratory drive. The maneuvers may be coached maneuvers requiring a clinician to coach the patient to perform the maneuver, or alternatively, the ventilator 202 may coach the patient to perform the maneuver without clinician assistance. For example, ventilator 202 may provide a patient with audio, visual, and/or tactile indications to perform a maneuver.

Weaning readiness detection module 224 may initiate any of the above mentioned maneuvers when measured or calculated patient data meets specific conditions, for example, when ventilatory parameters meet a specified maneuver criteria. For example, when a patient has been on a low level of ventilator support and is spontaneously triggering breaths without frequent periods of apnea, and the patient's RSBI, hemodynamic measurement and oxygenation status are in an acceptable range or meet specific threshold criteria, one or more of the above mentioned maneuvers may be performed to determine weaning readiness. As should be appreciated, with reference to the Equation of Motion, ventilatory parameters are highly interrelated and, according to embodiments, may be either directly or indirectly monitored. That is, parameters may be directly monitored by one or more sensors, as described above, or may be indirectly monitored by derivation according to the Equation of Motion.

Weaning readiness detection module 224 may also or alternatively collect, analyze and trend external patient data from external devices 230 via integrated network module 228 to determine whether a patient is ready for a weaning trial. For example, weaning readiness detection module 224 may base a determination of weaning readiness on external data such as end-tidal CO₂, SpO₂, level of sedation, brainwave activity, blood oxygen level, cardiac output, blood pressure, prescriptions or other medications (e.g., blood pressure medication, etc), or other parameters that are relevant to a patient's readiness to undergo a weaning trial. In particular, for example, a patient experiencing a threshold level of sedation, a patient having a low blood oxygen level, or a patient on blood pressure medication, would not be a good candidate for weaning trials. For example, a patient having a rating of about 3 to about 4 on the Riker Sedation scale or a reading greater than about 70 on a bispectral index (BIS) monitor would indicate that the patient is not a good candidate for weaning trials.

In some embodiments, both the internal and external patient data parameters described above may be integrated together by weaning readiness detection module 224 for a combined determination of weaning readiness. For example, a patient having a combination of a dead space to tidal volume ratio (V_{d}/Vₜ) of less than about 45%, a static compliance greater than about 0.6 mL/cm H₂O/kg, a RSBI less than about 105, and a PaCO₂ less than about 50 mmHg may be a suitable candidate for a weaning trial.

A clinician may select specific internal and/or external patient data parameters and combine or group the selected patient data parameters into a weaning readiness parameter package for use by ventilator 202 to determine the weaning readiness of a patient. Alternatively, one or more predefined weaning readiness parameter packages may be supplied with the ventilator 202 from the manufacturer. The predefined weaning readiness parameter packages may define the specific threshold values of each parameter for determining the weaning readiness of a patient. As an example, each weaning readiness parameter package may be directed for use with patients having a particular disease or other similar respiratory problems. For example, a weaning readiness threshold value corresponding to compliance may be set lower for a patient with fibrosis (lower than average compliance) since it is unlikely that a patient with fibrosis would have a compliance high enough to reach a normal or nominal compliance threshold. The predefined weaning readiness parameter packages may also be adjustable or modifiable by the clinician to allow for tailoring to the specific needs of each patient. For example, the clinician may modify or adjust the threshold for each patient parameter to tailor the weaning readiness package to a particular patient.

In some embodiments, weaning readiness detection module 224 may weight each patient parameter based on a level of importance to the determination of a patient's weaning readiness. For example, if parameters having a higher weighting meet the threshold criteria while parameters having a lower weighting do not meet threshold criteria, weaning readiness detection module 224 may still make a determination of weaning readiness based on the relative importance of each patient parameter to weaning readiness. Patient parameters such as, for example, compliance and resistance, may have higher weighting than patient parameters such as flow, volume, respiratory frequency, etc. As an example, elevated levels of PaCO₂ may be weighted less for a patient with COPD that has a known elevated CO₂ retention than for a patient having a normal CO₂ retention.

Weaning readiness detection module 224 may employ a weaning readiness matrix which includes the threshold values for each patient parameter and also includes conditions under which a grouping of patient parameters may allow for a determination of weaning readiness even though some or all of the patient parameters may not meet their threshold value. The weaning readiness matrix may be part of a weaning readiness package and may be modified or set by the clinician. For example, the matrix may include a combination of dead space to tidal volume ratio, static compliance, RSBI, PaCO₂, airway resistance, indices of muscle strength, and/or any other suitable respiratory parameters.

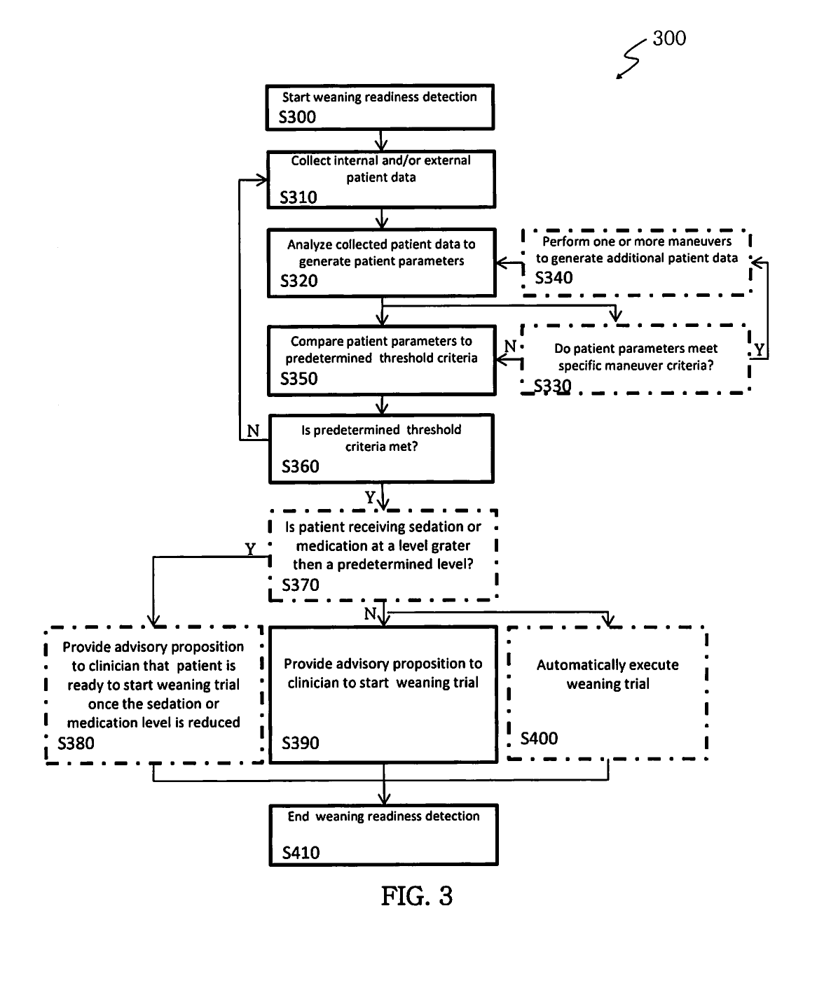

Referring now to FIG. 3, a method 300 of determining weaning readiness is illustrated. In step S300, the weaning readiness detection module 224 starts weaning readiness detection. Next, in step S310, the weaning readiness detection module 224 collects the internal and/or external patient data from distributed sensors 218, internal sensors 220, memory 208, external devices 230 and/or other sources of patient data as described above.

In step S320, the weaning readiness detection module 224 analyzes the collected internal and/or external patient data to generate patient parameters such as, for example, respiratory mechanics, indices of muscle strength, etc.

In some embodiments, a step S330 may be included in which weaning readiness detection module 224 compares the generated patient parameters to specific maneuver criteria, for example, RSBI less than about 105, P_{0.1} equal to about 2-6 cm H₂O, Vₜ greater than about 3 mL/kg, V_{d}/Vₜ less than about 40%, and/or MIP greater than about 20 cm H₂O. If the specific maneuver criteria is met, execution proceeds to S340, otherwise execution proceeds to S350. In step S340, if the specific maneuver criteria is met, weaning readiness detection module 224 may trigger one or more maneuvers (e.g., P_{0.1}, VC, NIF, etc.) to generate additional measurable patient data from the patient. Once the maneuvers are complete, execution proceeds to step S320 and the additional patient data is analyzed. In some embodiments, the maneuvers may be automatically activated during step S310.

In step S350, the weaning readiness detection module 224 performs a closed-loop decision making process based on the patient parameters by comparing the patient parameters with predetermined threshold criteria. For example, weaning readiness may be determined based solely on a predetermined threshold criteria of a single parameter, or may be determined based on a predetermined threshold criteria for a combination of patient parameters.

In step S360, the weaning readiness detection module 224 determines if the predetermined threshold criteria is met. For example, weaning readiness detection module 224 may determine if resistance is less than a predetermined resistance threshold, compliance is greater than a predetermined compliance threshold, the indices of muscle strength are greater than a predetermined muscle strength threshold, and/or any other patient parameters and threshold criteria. Some Examples of threshold criteria include a MIP greater than about 20 cm H₂O, a RSBI less than about 105, respiratory rate less than about 30/min, SpO2 greater than about 90% on less than about 50% O₂, and Vₜ greater than about 3 mL/kg

In some embodiments, a step S370 may be included in which, if the predetermined threshold criteria is met, the weaning readiness detection module 224 further determines whether the patient is receiving sedation or a particular medication at a level greater than a predetermined level. In step S380, if the patient is receiving sedation or medication at a level greater than the predetermined level, an advisory proposition is provided to the clinician that the patient is ready to start weaning trials once the sedation or medication level is reduced, e.g., via display module 204, user interface 210, smart prompt module 226, a hand held device, a nursing station, a hospital server, a central monitoring station or another location set by a clinician. Execution then proceeds to step S410 and terminates.

In step S390, if the predetermined threshold criteria is met (step S360), and in some embodiments, if the patient is not receiving sedation or medication at a level greater than the predetermined level (step S370), the weaning readiness detection module 224 provides an advisory proposition to the clinician that the patient is ready for weaning trials, e.g., via display module 204, user interface 210, smart prompt module 226, a hand held device, a nursing station, a hospital server, a central monitoring station or another location set by a clinician. Execution then proceeds to step S410 and terminates.

In step S400, alternatively or in addition to step S390, if the predetermined threshold criteria is met (step S360), and in some embodiments, if the patient is not receiving sedation or medication at a level greater than the predetermined level (step S370), the weaning readiness module 224 commands the ventilator 202 to automatically execute weaning trials. Execution then proceeds to step S410 and terminates. In some embodiments, if the weaning trial is successful, e.g., spontaneous breathing is successful for a certain predefined period of time such as half an hour, an hour, etc., weaning readiness detection module 224 may further provide the clinician with an advisory proposition noting the success of the weaning trials and that removal of the ventilation tubing system 130 is now appropriate.

If the predetermined threshold criteria is not met (S360), execution returns to step S310 and repeats.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure.

The foregoing examples illustrate various aspects of the present disclosure and practice of the methods of the present disclosure. The examples are not intended to provide an exhaustive description of the many different embodiments of the present disclosure. Thus, although the foregoing present disclosure has been described in some detail by way of illustration and example for purposes of clarity and understanding, those of ordinary skill in the art will realize readily that many changes and modifications may be made thereto without departing form the spirit or scope of the present disclosure.

While several embodiments of the disclosure have been shown in the drawings and described in detail hereinabove, it is not intended that the disclosure be limited thereto, as it is intended that the disclosure be as broad in scope as the art will allow. Therefore, the above description and appended drawings should not be construed as limiting, but merely as exemplifications of particular embodiments. Those skilled in the art will envision other modifications within the scope and spirit of the claims appended hereto.

What is claimed is:

1. A method of detecting weaning readiness of a patient comprising:
collecting patient data;
analyzing the collected patient data to generate at least one patient parameter;
comparing the at least one patient parameter to predetermined weaning readiness threshold criteria; and
providing an advisory proposition to a clinician indicating a readiness of the patient to start a weaning trial if the at least one patient parameter meets the weaning readiness threshold criteria.

2. The method according to claim 1, further including automatically executing a weaning trial if the at least one patient parameter meets the weaning readiness threshold criteria.

3. The method according to claim 1, wherein the patient data includes patient data collected by a ventilator.

4. The method according to claim 1, wherein the patient data includes patient data collected by a device selected from the group consisting of capnometers, pulse oximeters, brain function monitors and hemodynamic monitors.

5. The method according to claim 1, wherein the at least one patient parameter is selected from the group consisting of a respiratory compliance of the patient, respiratory resistance of the patient and an index of muscle strength of the patient.

6. The method according to claim 5, wherein the predetermined weaning readiness threshold criteria is met when at least one of the compliance of the patient is greater than a predetermined compliance threshold, the resistance of the patient is less than a predetermined resistance threshold, and the index of muscle strength of the patient is greater than a predetermined muscle strength threshold.

7. The method according to claim 1, further including providing a weaning readiness parameter package including at least two patient parameters, wherein the predetermined weaning readiness threshold criteria is met when the parameters in the weaning readiness parameter package meet a predetermined parameter package threshold.

8. The method according to claim 7, wherein the parameters in the weaning readiness parameter package are weighted based on a relative importance to a weaning readiness of the patient.

9. The method according to claim 8, wherein the predetermined parameter package threshold is met when at least one parameter of the weaning readiness parameter package having a higher weight relative to at least one other parameter of the weaning readiness parameter package meets a predetermined parameter threshold.

10. The method according to claim 1, further comprising performing one or more maneuvers on the patient to generate additional patient data if the patient parameters meet specific maneuver criteria.

11. The method according to claim 1, further comprising:
determining if the patient is receiving a specified medication at a level greater than a predetermined level; and
providing an advisory proposition to a clinician indicating a readiness of the patient to start a weaning trial once the level of the specified medication that the patient is receiving is reduced below the predetermined level, if the at least one patient parameter meets the weaning readiness threshold criteria and the patient is receiving sedation at a level greater than or equal to the predetermined level.

12. The method according to claim 11, wherein the specified medication is a sedative.

13. A non-transitory computer-readable storage medium encoded with a program that, when executed by a processor detects weaning readiness of a patient and causes the processor to perform the steps of:
collecting patient data;
analyzing the collected patient data to generate at least one patient parameter;
comparing the at least one patient parameter to predetermined weaning readiness threshold criteria; and
providing an advisory proposition to a clinician indicating a readiness of the patient to start a weaning trial if the at least one patient parameter meets the weaning readiness threshold criteria.

14. The non-transitory computer-readable storage medium according to claim 13, further performing the step of:
automatically executing a weaning trial if the at least one patient parameter meets the weaning readiness threshold criteria.

15. The non-transitory computer-readable storage medium according to claim 13, wherein the at least one patient parameter is selected from the group consisting of a respiratory compliance of the patient, respiratory resistance of the patient and an index of muscle strength of the patient.

16. The non-transitory computer-readable storage medium according to claim 15, wherein the predetermined weaning readiness threshold criteria is met when at least one of the compliance of the patient is greater than a predetermined compliance threshold, the resistance of the patient is less than a predetermined resistance threshold, and the index of muscle strength of the patient is greater than a predetermined muscle strength threshold.

17. A ventilatory system for detecting weaning readiness of a patient comprising:
a ventilator;
at least one sensor disposed in communication with the ventilator;
a processor disposed in communication with the at least one sensor and the ventilator, the processor configured to collect patient data from the at least one sensor and to analyze the collected patient data to generate at least one patient parameter, the processor further configured to compare the at least one patient parameter to a predetermined weaning readiness threshold criteria; and
an output device configured to display an advisory proposition to a clinician indicating a readiness of the patient to start a weaning trial if the at least one patient parameter meets the weaning readiness threshold criteria.

18. The ventilatory system according to claim 17, wherein the processor is configured to automatically command the ventilator to execute a weaning trial if the at least one patient parameter meets the weaning readiness threshold criteria.

19. The ventilatory system according to claim 17, wherein the processor is remote from the ventilator.

20. The ventilatory system according to claim 17, wherein the processor is configured to automatically command the ventilator to perform one or more maneuvers on the patient to generate additional patient data if the at least one patient parameter meets specific maneuver criteria.
